(19) United States
(12) Patent Application Publication
(10) Pub. No.: US 2020/0143925 A1
(43) Pub. Date: May 7, 2020

(54) FORENSIC SYSTEM AND METHOD FOR DETECTING FRAUD, ABUSE, AND DIVERSION IN THE PRESCRIPTIVE USE OF CONTROLLED SUBSTANCES

(71) Applicant: Timothy Earl King, Carmel, IN (US)

(72) Inventor: Timothy Earl King, Carmel, IN (US)

(21) Appl. No.: 16/666,971

(22) Filed: Oct. 29, 2019

Related U.S. Application Data
(60) Provisional application No. 62/755,605, filed on Nov. 5, 2018.

(51) Int. Cl. G16H 20/10; G16H 10/60; G16H 15/00

(52) U.S. Cl. CPC G16H 20/10; G16H 15/00; G16H 10/60

(57) ABSTRACT

The present invention relates to a forensic system and method, including a methodology, for analyzing medical and pharmacy data to determine the legitimacy of controlled substance prescription and use, and to detect fraud, abuse, and/or diversion of controlled substances. In one aspect, said method provides objective evidentiary data that shows with high certainty whether a suspected medical practitioner has been issuing controlled substance prescriptions “outside the usual course of medical practice” and/or “for other than legitimate medical purposes”. Said method rearranges, organizes, and simplifies the complex data in patients' medical and PDMP charts, and provides a series of three sequential work products in spreadsheet form, a Forensic Chronology chart, a Forensic Summary chart, and a Standard of Care Summary chart, via which medical data is effectively translated into objective criteria from which a legal conclusion can be determined by legal scholars, prosecutors, defense attorneys, and juries.

### CROSS-REFERENCE TO RELATED APPLICATIONS

This application claims priority under 35 USC § 119(e) to U.S. Provisional Patent Application Ser. No. 62/755,605, filed on Nov. 5, 2018, the disclosure of which is incorporated herein by reference in its entirety.

### FIELD OF INVENTION

The present invention relates to a forensic system and methodology for review of controlled substance use, and, more specifically, to a forensic system and method for analyzing medical and pharmacy data to determine the legitimacy of controlled substance use, in order to detect fraud, abuse and/or diversion of controlled substances.

### BACKGROUND OF INVENTION

Although the invention disclosed in the following pertains primarily to a system and method for detecting the proper use of controlled substances, such as opioids, stimulants, and sedatives, it is to be understood that, as contemplated herein, the invention may have usefulness in the detection of proper medical use of a broad variety of other legend drugs. Legend drugs are all substances that require a prescription for legal possession, e.g., antibiotics, blood pressure drugs, pain medications, and the like. Legend drugs are inclusive of controlled substances, which require additional certification from the Drug Enforcement Administration (DEA) in order to be legitimately prescribed.

The DEA put out a publication in 2006 called the Practitioner's Manual, intended for the purpose of educating physicians and other medical practitioners in the rules by which they were to prescribe controlled substances, such as opiates, for a legitimate medical purpose in a manner consistent with the practice of medicine. An additional purpose was to minimize the illegal “pill mill” approach to the use of controlled substances. The DEA is an agency of the Department of Justice (DOJ) of the United States federal government. It is an enforcement agency, and thus its role is limited to enforcement, as defined by the 1973 Controlled Substances Act (CSA) established by Congress. As such, the DEA does not regulate medicine, and is not in a position to decide, nor is it allowed to determine, whether or not prescriptions are being issued by a provider for a legitimate medical purpose. The CSA has also defined what is called a “Closed System of Distribution”, under which all legitimate handlers of controlled substances (e.g., doctors, pharmacies, hospitals, researchers, distributors, manufacturers, and the like), have to be registered with the DEA, with the intent that all controlled substances would be followed from handler to user in a manner that would have accountability and would diminish illegitimate use. The Practitioner's Manual defines the key parameters within which controlled substances are to be prescribed, and stipulates that, for a prescription for a controlled substance to be valid, the prescription must be issued for a legitimate medical purpose by a practitioner acting in the usual course of professional practice. Accordingly, phrases such as “for a legitimate medical purpose”, and “in the course of usual medical practice”, have become key criteria in determining whether a certain prescription is being issued legitimately, and serve as the foundation upon which criminal prosecution of illegitimate prescribers of controlled substances are made. In other words, if a prescription is not issued in the usual course of professional treatment for a legitimate and authorized purpose, it is not a valid prescription; and upon that rests the criminal prosecution of the prescriber.

The abuse of prescription drugs and controlled substances, particularly opiates, has grown substantially over the years, and has become a problem of epidemic proportions in today's society. A recent report from the National Safety Council states: “The opioid crisis is worsening. Over 42,000 Americans died of an opioid overdose in 2016, and government and public health officials are scrambling to find effective ways to reverse this frightening trend. As the death toll from opioid overdose increases, addressing the crisis becomes ever more urgent.”

Controlled substances are defined in the DEA Practitioner Manual according to five major classifications, Schedule 1 to Schedule 5. The classification of a controlled substance is based on 2 factors: medication safety and generally accepted medical use. For example, heroin is a schedule 1 drug because it is highly addicting and has no generally accepted medical use. Schedule V drugs are defined as drugs with lower potential for abuse than Schedule IV and consist of preparations containing limited quantities of medically accepted narcotics, such as codeine cough syrups. However, while federal law dictates that each controlled substance must be prescribed by a medical practitioner “for a legitimate medical purpose,” yet neither the US Congress, nor the DEA, nor the DOJ have further defined what is meant by “legitimate medical purpose.” Thus, the definition of “other than legitimate use” of a controlled substance has ultimately fallen to the medical expert, based on professional qualifications, practice expertise, and objective methodology.

Since the practice of medicine is carried out strictly under the purveyance of medical practitioners practicing under state medical board jurisdiction, the determination of medical legitimacy falls to the opinion of medical experts willing to testify in court as to the medical appropriateness (e.g., legitimacy) of controlled substance use. Nonetheless, criminal prosecution of medical practitioners that issue controlled substances without a legitimate medical purpose is a collaborative effort between governmental agencies and medical experts. However, in relying upon the testimony of medical expert witnesses, it is not sufficient to rely merely on subjective opinions of said expert witnesses. It is required that medical experts thoroughly understand standards of medical care, and are thus able to provide objective testimony based on defined methodology, under oath, as to whether controlled substances (e.g., narcotics, stimulants, sedatives, and the like) are prescribed for a legitimate medical purpose and in the usual course of medical practice.

Determining whether the controlled substances are being prescribed for a legitimate medical purpose and within the usual course of medical practice is a complex matter. There are no objective “switches”, defined sets of criteria, or generally accepted medical protocols that conclude whether a specific medical practitioner has issued a controlled substance prescription outside the usual course of medical practice (i.e., pill mill operation), or for a legitimate medical purpose (i.e., for purposes of fraud, abuse, or diversion). Considering the serious nature of criminal prosecution, i.e., accusations that lead to criminal conviction of the medical practitioner, forfeiture of medical licensure, loss of freedom (prison), seizure of monetary and property assets, and seizure of all items attributable to the illegitimate prescribing of the controlled substances—the courts understandably do not take a favorable view of an expert witness providing a biased, subjective, or overly simplified opinion on the matter. Rather, it is required that the expert witness provide objective testimony, according to defined methodology, with supporting data, showing beyond a reasonable doubt, e.g., with 99% certainty, that the medical practitioner being investigated has indeed prescribed controlled substances “outside the usual course of medical practice and for other than a legitimate medical purpose”. Typically, to be admitted to court as an expert witness, Federal Rule of Evidence 702 requires that expert witnesses have “knowledge, skill, experience, training, or education” which will “help the trier of fact understand the evidence and determine a fact in issue.” Additionally, the expert witness is expected to lay objective and convincing groundwork, based on defined methodology, to support testimony, beyond a reasonable doubt, that the medical practitioner being investigated prescribed controlled substances outside the usual course of medical practice and for other than a legitimate medical purpose. Moreover, any evidentiary data provided by the expert witness would need to stand up to a “Daubert challenge” that an opposing counsel may present. The Daubert challenge is a hearing conducted before a judge where the validity and admissibility of expert testimony is challenged by opposing counsel. The expert witness is required to demonstrate that the methodology and logic used in the case are scientifically valid and are applicable to the facts. (See: https://en.wikipedia.org/wiki/Daubert standard). If the expert witness cannot put forth or defend a scientifically valid methodology as required by the Daubert challenge, then the expert's testimony can be excluded from the courts. The critical importance of having highly qualified, credible expert witnesses who provide medical-legal conclusions based on objective and scientifically based methodology cannot be overemphasized. Too many times practitioners are willing to provide “opinions for hire”, a practice that degrades efforts to support the justice system in a credible, professional, and helpful manner.

In view of the foregoing, especially regarding the urgent need to combat and curtail the epidemic of prescription drug abuse, a forensic product is needed that provides an objective, data-based methodology based on a clear set of criteria, to help determine if a medical practitioner suspected of promoting fraud, abuse, or diversion has issued controlled substance prescriptions for a legitimate medical purpose. In other words, such a forensic product and methodology would need to show objectively, without a reasonable degree of doubt, whether prescriptions for said controlled substances were issued for “a legitimate medical purpose by a practitioner acting in the usual course of professional practice”.

Until now, the Applicant of the invention herein (hereinafter, “the Applicant”) is not aware that such an objective method based on data has been developed or that is available, or of any existing product that helps the expert witness to scientifically investigate and lay out the evidentiary groundwork in an objective and reproducible manner. In the following, the Applicant discloses the invention of precisely such a product that provides the desired objective and reproducible methodology. The Applicant himself is a highly qualified medical expert (QME) witness, being a medical doctor with several decades of medical practice, including in the fields of anesthesiology, pain management, and addiction. Additionally, the Applicant has a lengthy record of working with the DEA, Federal Bureau of Investigation (FBI), Food and Drug Administration (FDA), Department of Justice (DOJ), municipal/state courts, and state medical boards. He aids in the criminal prosecution of pill mills and over-prescribing practitioners suspected of issuing controlled substance prescriptions “outside the usual course of medical practice”, and “for other than legitimate medical purposes”.

### SUMMARY OF INVENTION

In one embodiment, the present disclosure provides a reproducible method useful for objectively determining the medical legitimacy of prescriptions issued for controlled substances. In one aspect, said method provides objective evidentiary data that shows with high certainty whether a suspected medical practitioner has been issuing controlled substance prescriptions “outside the usual course of medical practice” and/or “for other than legitimate medical use”. In another aspect, said method has resulted in a work product, in three parts, that reduces the subjectiveness that often accompanies complex scientific opinions, by translating medical data (e.g., patient medical records, patient prescription drug data, and other patient data) into objective criteria from which a legal conclusion can be derived. Legal scholars, prosecutors, defense attorneys, juries, and the like, require a summarization of complex medical data from which a legal conclusion can be determined; the work product produced by said method herein provides the required summarization of complex medical data from which a legal conclusion can be made, especially in cases involving suspected issuance of controlled substance prescriptions “outside the usual course of medical practice” and/or “for other than legitimate medical use”. Said work product is derived via analyses of patients' medical charts.

In another embodiment, the aforementioned reproducible and objective method provides a series of three sequential work products that significantly reduce the subjectiveness that necessarily accompanies complex scientific/medical opinions, by effectively translating medical data into objective criteria from which a legal conclusion can be achieved. The said three sequential work products are described herein by the following terminology: (1) Forensic Chronology; (2) Forensic Summary; and, (3) Standard of Care Summary. This series of three work products achieves the goal of analyzing and summarizing complex medical data from which a legal conclusion can be determined by legal scholars, prosecutors, defense attorneys, and juries. All data is source-based to the medical chart. Each of the three sequential work products builds on data and conclusions derived from the previous work product. The logical progression of analysis concludes with an objective summarization as to whether medication was prescribed for a legitimate medical purpose according to generally accepted medical standards of care.

In one embodiment, said Forensic Chronology is provided for a particular patient from a group of patients of a particular medical provider, which comprises a summary of relevant medical chart data that has been integrated with the patient's prescription drug data that is obtainable from the prescription data monitoring program (PDMP), so as to present a chronological overview/summary of prescribed drugs, clinical rationale, diagnostic foundation, past medical history, and clinical response. A separate Forensic Chronology document is provided for each patient in said group of patients. The method used to develop the Forensic Chronology work product involves the use of highly developed and technically applied tools in an Excel spreadsheet, as is described in detail in the Detailed Description section below. All data is chronologically ordered, color coded, and source documented. In one key aspect, the data in said Forensic Chronology spreadsheet has been color coded in order to discern key medical data, events, and patterns of drug use. Color differentiation allows for quick identification of doctor-shopping, inappropriate polypharmacy, and aberrant patient behavior. Illustratively, prescribed medications and office visits by the target provider are colored blue; other-provider consultations and prescriptions are colored pink. Other entries have additional color coding, e.g., green for urine drug screens (UDS), purple for imaging, white for nursing notes, and orange for hospital and ER visits. All material pertinent to establishment of a diagnosis, examination of the patient, formulation of a treatment plan, and patient accountability are easily visualized in a chronological format. Additionally, demographic data and relevant clinical events are documented in header format at the beginning of the Forensic Chronology spreadsheet document. All data is sourced from the medical chart by reference to Bates-stamped entries electronically printed on each medical chart page. Any observation, test, diagnosis, consultation, or prescription can be easily and objectively verified by going back to the appropriate page in the medical chart. Each patient has a separate Forensic Chronology. There is a section delineating pertinent demographic information and critical clinical findings. Judgements of clinical adequacy are made in this section based on observations including clinical exam sufficiency, independent medical evaluation, aberrant patient behavior enforcement, testing and documentation adequacy. These observations provide critical foundation for the next product in the series of three products, the aforementioned Forensic Summary. Further details on the development of the Forensic Chronology document are described in the Detailed Description section below.

In another embodiment, said Forensic Summary is provided, which is the second in said series of three sequential work products. It comprises a succinct summarization of relevant data that is gleaned from the patients' Forensic Chronology documents described above for the particular medical provider. It is likewise presented in spreadsheet format, so as to facilitate review and interpretation, via which a medical-legal opinion can be derived. The data is arranged according to multiple criteria that define the ultimate legitimacy of controlled substance use, and as well is color-coded in a manner that simplifies review and interpretation. In the Forensic Summary, a hierarchal approach first defines six (6) medical standards of care: (1) Medical diagnosis; (2) Support of diagnosis; (3) Risk assessment; (4) Treatment plan; (5) Procedures; and, (6) Compliance/Enforcement/Outcome. Standards of care are further subdivided into fifteen (15) applicable care standards for the use of controlled substances. Controlled substance standards are subdivided into 15 targeted queries of the medical chart. Standards of care, Opiate Parameters, and Targeted Queries ultimately represent 15 specific medical-legal criteria that form the foundation of “legitimate medical use”. Further details regarding the development of the Forensic Summary document are described in the Detailed Description section below.

In another embodiment, said Standard of Care Summary mentioned above is provided, which is the third in the foregoing series of three sequential work products, and it rates all standards of care criteria as having been met or unmet. It is typically a one-page product that comprises a concise spreadsheet summary of the Forensic Summary described above, expressed as a series of notations representing whether parameters (Targeted Queries) listed under the Standards of Care have been legally fulfilled. Each parameter is given a single designation:

| | |
|---|---|
| ✓ | The Standard of Care is fulfilled. |
| ◯ | Documentation is lacking. |
| X | The Standard of Care is violated. |
| XX | An egregious violation of the Standard of Care is observed. |

Thus, the Standard of Care Summary for a particular medical provider provides a concise, one-page document from which a legal determination can be made as to whether federal or state laws have been violated regarding the appropriate use of controlled substances. It clearly indicates whether medication is not prescribed in a legitimate medical manner if standards of care are not met. Further details on the development of the Standard of Care Summary document are described in the Detailed Description section below.

The foregoing embodiments of the invention, and additional embodiments, are described in greater detail in the Detailed Description section below.

All publications, if any, cited throughout this application are incorporated herein by reference in their entirety. Indeed, throughout this description, it is to be understood that any and all publicly available documents described herein, including any and all cited U.S. patents, patent applications, and non-patent publications, are specifically incorporated by reference herein in their entirety. Nonetheless, the related art and publications described herein are not intended in any way as an admission that any of the documents described therein, including pending U.S. patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

Naturally, further objects, advantages and features of the present invention are disclosed throughout other areas of the specification, and will become apparent from the following detailed description and claims.

### DESCRIPTION OF DRAWINGS

The foregoing summary, as well as the following Detailed Description of the preferred embodiments of the invention, will be better understood when read in conjunction with the accompanying figures, in which:

FIG. 1 depicts an illustrative, non-limiting example of a representative Forensic Chronology chart of the invention.

FIG. 2 depicts an illustrative, non-limiting example of a representative Forensic Summary chart of the invention.

FIG. 3 depicts an illustrative, non-limiting example of a representative Standard of Care Summary chart of the invention.

### DETAILED DESCRIPTION

Before the present details of the invention are disclosed and described, it is to be understood that this invention is not limited to the specific components, methods, and implementation, or to the precise arrangements and instrumentalities shown, as such may, of course, vary while remaining within the scope and spirit of the invention. It is also to be understood that the terminology used herein is for the purpose of describing particular implementations only, and to assist in understanding the disclosure, and is not intended to be limiting.

In one embodiment, the present disclosure provides a reproducible method useful for objectively determining the medical legitimacy of prescriptions issued for controlled substances. In one aspect, said method provides objective evidentiary data that shows with high certainty whether a suspected medical practitioner has been issuing controlled substance prescriptions “outside the usual course of medical practice” and/or “for other than legitimate medical use”. In another aspect, said method has resulted in a work product, in three parts, that reduces the subjectiveness that often accompanies complex scientific opinions, by translating medical data (e.g., patient medical records, patient prescription drug data, and other patient data) into objective criteria from which a legal conclusion can be derived. Legal scholars, prosecutors, defense attorneys, juries, and the like, require a summarization of complex medical data from which a legal conclusion can be determined; the work product produced by said method herein provides the required summarization of complex medical data from which a legal conclusion can be made, especially in cases involving suspected issuance of controlled substance prescriptions “outside the usual course of medical practice” and/or “for other than legitimate medical use”. Said work product is derived via analyses of patients' medical charts. The medical chart, by definition, contains the entirety of data that went into decision making by the medical provider. It is a complete record of a patient's key clinical data and medical history, including demographics, vital signs, diagnoses, medications, treatment plans, progress notes, problems, immunization dates, allergies, radiology images, and laboratory and test results. Past treatment trials, phone conversations, nursing notes, pharmacy notifications, insurance letters, third party conversations, hospital and ER notifications would also be included in the record. In an ideal world, the medical chart could be “data-mined” for key information that would ultimately support, or fail to show support for, the use of prescriptive medications. In order to effectively analyze the medical chart in a practical and time effective manner, specific criteria would need to be defined. Standards of care would need to be acknowledged. An objective record of pertinent data would need to be succinctly presented so that conclusion of medical legitimacy could ultimately be derived.

In another embodiment, the aforementioned reproducible and objective method, which comprises a comprehensive review of the medical chart, provides a series of three sequential work products that significantly reduce the subjectiveness that necessarily accompanies complex scientific/medical opinions, by effectively translating medical data, i.e., patient medical record, into objective criteria from which a legal conclusion can be achieved. Key concepts are objectively and clearly defined by this method, such as: (a) Is there medical support for the legitimate use of controlled substances? (b) Do aberrant patterns of medical practice exist? (c) Is there evidence for fraud? Accordingly, the said three sequential work products are described herein by the following terminology: (1) Forensic Chronology; (2) Forensic Summary; and, (3) Standard of Care Summary. This series of three work products achieves the goal of analyzing and summarizing complex medical data from which a legal conclusion can be determined by legal scholars, prosecutors, defense attorneys, and juries. All data is source-based to the medical chart. Each of the three sequential work products builds on data and conclusions derived from the previous work product. The logical progression of analysis concludes with an objective summarization as to whether medication was prescribed for a legitimate medical purpose according to generally accepted medical standards of care.

In one embodiment, said Forensic Chronology is provided, which is the first in said series of three sequential work products, and comprises a summary of relevant medical chart data that has been integrated with the patient's prescription drug data that is obtainable from the prescription data monitoring program (PDMP), so as to present a chronological overview/summary of prescribed drugs, clinical rationale, diagnostic foundation, past medical history, and clinical response. Stated another way, said Forensic Chronology is a chronological summation of all events, doctor visits, hospitalizations, data, imaging, urine drug screens, and physician consultations; and the PDMP data, appropriately modified and translated into Excel spreadsheet format, is also incorporated into the chronology of medical care. The method used to generate the Forensic Chronology spreadsheet involves the use of sophisticated sorting, filtering, and data management tools, such as are available in the Excel program. Thus, in one variation of the process of the invention, all medical data is manually summarized and extracted from each patient's medical chart and placed (e.g., by “cutting and pasting” and/or transcribing) into an Excel spreadsheet, and all pertinent drug data for the same patient is manually summarized and extracted from the patient's PDMP and placed into a second Excel spreadsheet; these two data sets are then integrated into a single final Excel spreadsheet, as described below. Sort and Filter functions are then used to manipulate the data into an integrated series of chronological events to end up with the Forensic Chronology spreadsheet, thus demonstrating an integrated timeline of medical care and controlled substance prescribing. In a preferred variation of the process of the invention, a “skeleton” Forensic Chronology Excel spreadsheet is first created (see below for examples), and the data from the medical chart is input directly into it manually; then the drug data from the PDMP is input into it, either directly or via a second, preliminary Excel spreadsheet. This is followed by sorting/filtering, to end up with the Forensic Chronology spreadsheet. In one key aspect, the data in said Forensic Chronology spreadsheet may be coded for further analysis by any of a variety of coding methods known in the art. For the purpose of the invention herein, color coding has been found to be an effective and preferred visual method that simplifies review and interpretation, and offers easy recognition and differentiation of specific events. Thus, color coding is then applied to each entry in said Forensic Chronology spreadsheet to demonstrate the type of clinical event: e.g., office visit, hospitalization, drug test, imaging result, medical consultation, prescription, nursing notes, etc. Illustratively, for the purpose of the invention herein, prescribed medications and office visits by the target medical provider are colored blue; other-provider consultations and prescriptions are colored pink. Color differentiation allows for quick identification of doctor shopping, inappropriate polypharmacy, and aberrant patient behavior. Other entries have additional color coding, e.g., green for UDS, purple for imaging, white for nursing notes, and orange for hospital and ER visits. All material pertinent to establishment of a diagnosis, examination of the patient, formulation of a treatment plan, and patient accountability are easily visualized in this chronological format. Additionally, demographic data and relevant clinical events are documented in header format at the beginning of the Forensic Chronology spreadsheet document. All data for each Forensic Chronology document of a certain patient is sourced from the medical chart of that patient by reference to Bates-stamped entries electronically printed on each medical chart page. Any observation, test, diagnosis, consultation, or prescription can be easily and objectively verified by going back to the appropriate page in the medical chart. This source documentation ensures objectivity and accuracy. Each patient has a separate Forensic Chronology. There is a section in the Forensic Chronology spreadsheet delineating pertinent demographic information and critical clinical findings. Judgments of clinical adequacy are made in this section based on observations including clinical exam sufficiency, independent medical evaluation, aberrant patient behavior enforcement, testing and documentation adequacy. These judgments are necessarily made by the qualified medical expert (referred to hereinafter as “QME”) overseeing the methodology described herein, to ensure accuracy and consistency and to minimize variation. These observations provide critical material for the next product in the series of three products, the aforementioned Forensic Summary. The Forensic Chronology for each patient can be as long as 100 pages in length or longer, depending on the length of the medical chart. Longer and more lengthy medical charts mean longer and more lengthy Forensic Chronologies. That is why, ultimately, the data in the Forensic Chronology documents pertaining to several patients of a particular targeted medical provider must be combined and summarized into a short and concise document, said Forensic Summary, summarizing critical elements of the patients' medical charts. To exemplify, attached to this disclosure are two actual multi-page, color-coded Forensic Chronology documents for two patients (out of a total of eight patients) obtained by the method herein, titled Addendum 1 and Addendum 2, wherein the real personal identifiers of the medical provider (referred to in the two addenda as “Dr. Y”) and the two patients (referred to as “Patient A” and “Patient B”) have been redacted to protect their privacy (see Addendum 1 and Addendum 2 attached herewith).

In another embodiment, said Forensic Summary is provided, which is the second in said series of three sequential work products. It comprises a succinct summarization of relevant data that is gleaned from the patients' Forensic Chronology documents described above for a particular targeted medical provider. It is likewise presented in spreadsheet format, so as to facilitate review and interpretation, via which a medical-legal opinion can be derived. The data is arranged according to multiple criteria that define the ultimate legitimacy of controlled substance use, and as well is color coded in a manner that simplifies review and interpretation. In the Forensic Summary, a hierarchal approach first defines six (6) medical standards of care, which are: (1) Medical diagnosis; (2) Support of diagnosis; (3) Risk assessment; (4) Treatment plan; (5) Procedures; and, (6) Compliance/Enforcement/Outcome. Standards of care are further subdivided into fifteen (15) applicable care standards for the use of opiates. Controlled substance standards are subdivided into 15 targeted queries of the medical chart. Standards of care, Opiate Parameters, and Targeted Queries ultimately represent 15 specific medical-legal criteria that form the foundation of “legitimate medical use”. Table 1 below summarizes the Forensic Summary Queries, based on six (6) Standards of Care, (15) Opiate Parameters, and Subsequent Targeted Medical Chart Queries. To exemplify, attached to this disclosure is an actual, color-coded Forensic Summary document obtained by the method herein, titled Addendum 3, for a particular medical provider (referred to as “Dr. Y”), derived from the Forensic Chronology documents of eight patients, wherein the real personal identifiers of the medical provider (“Dr. Y”) and the eight patients (referred to as “Patient A” to “Patient H”) have been redacted to protect their privacy (see Addendum 3 attached herewith).

TABLE 1
Forensic Summary Queries

| 6 Standards of Care | 15 Opiate Parameters | Targeted Medical Chart Queries |
|---|---|---|
| 1. Medical diagnosis | Documentation of objective diagnosis | Legitimate & objective diagnosis or |
| | | subjective complaint? |
| | | Failure to define an objective pain |
| | | diagnosis? |
| | | Failure to perform an independent medical |
| | | exam? |
| 2. Support of Diagnosis | Pertinent Clinical History | Past medical history assumed or objectively |
| | | defined? |
| | | Past medical history obtained, |
| | | acknowledged, and considered? |
| | Targeted Physical Exam | Objective and targeted? |
| | | Repetitive or rote? |
| | | Electronic medical record? |
| | | Failure to perform independent medical |
| | | examination? |
| | Clinical Workup | Imaging, electro-diagnostics, lab studies, |
| | | 2nd opinions? |
| | | Evaluation supportive of diagnosis? |
| | | Failure to perform independent medical |
| | | examination? |
| 3. Risk Assessment | Mental Health Co-Morbidities | Under psychiatric care? |
| | | Alcohol, substance abuse, addiction |
| | | history? |
| | | Mental health diagnoses? |
| | | History of abuse (emotional, physical, |
| | | sexual)? |
| | | Psychiatric history inquired, acknowledged, |
| | | and considered? |
| | Medical Co-Morbidities | Risk factors for respiratory depression, |
| | | cardiac, & renal disease? |
| | | Treatment induced side effects? |
| | | Medical history inquired, acknowledged, |
| | | and considered? |
| 4. Treatment Plan | Defined Treatment Plan | Defined, assumed, or completely lacking? |
| | | Opiate-centric treatment plan - or |
| | | multidisciplinary & individualized? |
| | | Treatment default to opiates? |
| | High Risk Drugs or Drug Combinations | Drugs of high street value or high overdose |
| | | risk? |
| | | Dangerous drug combinations? |
| | MEQ > 100 (Morphine Equivalent Dose) | Dose/risk assessment? |
| 5. Procedures | Pain Procedures | Clinical indication? |
| | | Prior conservative treatment? |
| | | Excessive number of injections? |
| 6. Compliance/ | UDS (Urine Drug Screen) | Noncompliance? |
| Enforcement/Outcome | | Illegal drugs? |
| | PDMP (Prescription Drug Program) | Multiple pharmacies? |
| | | Multiple provider polypharmacy? |
| | | Doctor shopping? |
| | Clinical Improvement | Failure to enforce compliance? |
| | | Failure to demonstrate objective evidence |
| | | of improvement in VAS (visual analog |
| | | score) |
| | | QOL (quality of life) |
| | | Measurable, meaningful, & sustained |
| | | function |
| | Drug Risk Behaviors/Observations | Early refills, lost/stolen meds, missed |
| | | appointments |
| | | ER visits/hospitalizations |
| | | Arrests |
| | | Overdose events |
| | Death/Discharge | Duration of care? |
| | | Death event? |
| | | Failure to exercise opiate exit strategy? |

In another embodiment, said Standard of Care Summary mentioned above is provided, which is the third in the foregoing series of three sequential work products, and it rates all standards of care criteria in the Forensic Summary document as having been met or unmet. It is typically a one-page product that comprises a concise spreadsheet summary of the Forensic Summary described above, expressed as a series of notations representing whether parameters (Targeted Queries) listed under the Standards of Care (see Table 1) have been legally fulfilled. Each parameter is given a single designation:

| | |
|---|---|
| ✓ | The Standard of Care is fulfilled. |
| 0 | Documentation is lacking. |
| X | The Standard of Care is violated. |
| XX | An egregious violation of the Standard of Care is observed. |

Accordingly, the Standard of Care Summary provides a concise document from which a legal determination can be clearly made as to whether federal or state laws have been violated regarding the appropriate use of controlled substances. It clearly indicates whether medication is not prescribed in a legitimate medical manner if standards of care are not met. Attached to this disclosure is an actual Standard of Care Summary document (Addendum 4) obtained by the method herein, for the same “Dr. Y” above, derived from the Forensic Summary document (i.e., Addendum 3) of the same eight patients above (“Patient A” to “Patient H”) (see Addendum 4 attached herewith). Examination of this particular Standard of Care Summary in Addendum 4 indicates, without reasonable doubt, that “Dr. Y” has consistently not prescribed controlled substances in a legitimate medical manner that meets standards of care; i.e., that “Dr. Y” has been consistently issuing controlled substance prescriptions “outside the usual course” and/or “for other than legitimate medical use”.

In the following, the method of the invention disclosed herein is described stepwise in greater detail, as it would typically be carried out by a person, typically a QME, seeking to determine whether or not a certain targeted medical provider has been issuing controlled substance prescriptions “outside the usual course of medical practice” and/or “for other than legitimate medical use”, i.e., for “a legitimate medical purpose by a practitioner acting in the usual course of professional practice”. Said person is usually a qualified medical expert (QME) who thoroughly understands the generally accepted standards of medical care. The QME may be aided by one or more assistants working under the QME's supervision, particularly during the early, painstaking stages of transferring and inputting the extensive data and information in the medical charts and PDMP charts into the Forensic Chronology charts. Also, this QME is usually someone who is requested by the DEA, state law enforcement units, and/or state courts to carry out analytical research, provide objective evidentiary data, and participate in court proceedings as an expert witness.

First, the QME (and assistants) obtains the medical charts for a set of patients of a particular medical provider targeted for investigation, hereinafter referred to as “Dr. T”. These charts may be provided to the QME, for instance, by the government. In this case, the government usually would have had indications, e.g., via its internal processes, that some suspicious activity involving controlled substance prescriptions by Dr. T is going on. The government may have raided Dr. T's offices, obtained the patients' medical charts, selected the medical charts they would like to be reviewed and analyzed, and provided these to the QME. The number of selected patient medical charts can be anywhere from 10 to 50 charts or more.

Second, the QME obtains the PDMP (Prescription Drug Monitoring Program) data on each of the patients. The PDMP is a state program, and is a record of all the controlled substances the patient was prescribed by all doctors and/or providers (dentists, podiatrist, nurse practitioner, etc.) in that state. All medical providers have access to the PDMP, including the targeted Dr. T. The PDMP typically is produced in PDF format, but can be provided by the states in Excel spreadsheet format via conversion software. The PDMP layout may vary from state to state, which may require manipulation of the PDMP spreadsheet by the QME and assistants to convert it to the format of the Forensic Chronology spreadsheet or into a format that is amenable for transfer of the data into the Forensic Chronology spreadsheet. The skills and procedures required for manipulation of the Excel spreadsheet are standard, very well-known functionalities of the Excel program that are widely applied in a plethora of technical fields.

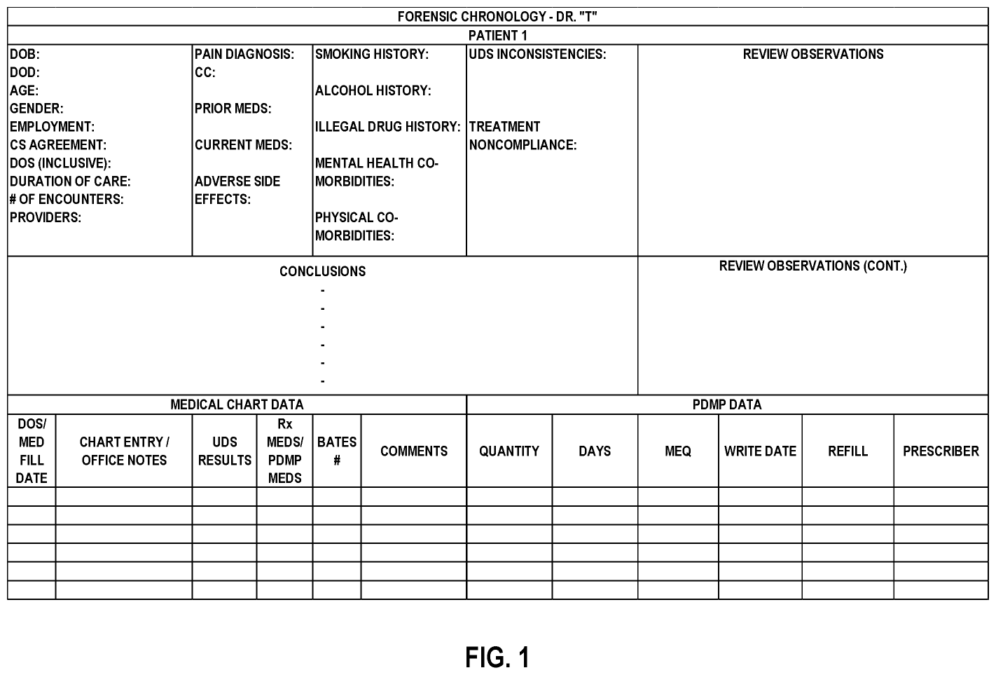

The QME (and/or assistants) creates for each patient an Excel spreadsheet that is a “skeleton” of the Forensic Chronology spreadsheet of the invention (i.e., that is consistent with the Forensic Chronology spreadsheet of the invention). A skeleton Forensic Chronology chart is displayed in FIG. 1 and in Addendum A attached herewith. (It is understood that the Forensic Chronology chart of the invention may have various layouts that are different than the one shown in FIG. 1 and Addendum A, but that would serve the same purpose).

Then, as stated above, all the medical data is manually summarized and extracted by the QME and/or assistants from the medical chart of each patient and placed into the skeleton Forensic Chronology Excel spreadsheet for that patient, and all pertinent drug data for each patient is manually summarized and extracted from the PDMP (which is typically a PDF document) and placed into a second Excel spreadsheet. Alternatively, the PDMP may already be obtained in Excel spreadsheet format, as mentioned earlier. The PDMP data for each patient is then integrated into the developing Forensic Chronology Excel spreadsheet for that patient, e.g., via “copy and paste” functionalities and/or transcription, to provide an integrated, single Excel spreadsheet for each patient. Then the data in this spreadsheet is rearranged and organized to appear chronologically by using the “sort” functionality of the Excel program; i.e., the spreadsheet is sorted by date, resulting in a final Forensic Chronology chart wherein the data appears as an integrated series of chronological events, thus demonstrating an integrated timeline of medical care and controlled substance prescribing, which constitutes the foundational data laid out in a manner that can be used by the QME as a reference point. It is understood that, as contemplated herein, several alternative Excel manipulating methods could be used by those skilled in the functionalities of the Excel program to arrive at the Forensic Chronology of the invention or an equivalent variant thereof. Integrating the patient's medical chart data and PDMP data into the skeleton Forensic Chronology chart is usually the “big” sub-step of the method herein, as it requires the QME (and/or assistants) to transfer the data from the typically very large medical chart (depending on how many years of records are obtained) to the Excel spreadsheet. The medical chart data is typically not displayed chronologically or in an orderly manner, so the QME (and/or assistants) has to painstakingly go through and pull out all the data (e.g., patient visits to Dr. T, visits to other doctor(s), hospitalizations, imaging studies, laboratory studies, urine drug tests, nursing notes, physical examinations, history, diagnoses, and all other data), read every single note and every single word, and parse this data into all the specific parts of the skeleton Forensic Chronology chart. This also includes all the demographic data in terms of age, number of visits, what the diagnoses were, what the medications were that were prescribed, arrests, notes from the court, any additional data supplied by the prosecutor, etc. Color coding of the PDMP and medical chart data being integrated into the skeleton Forensic Chronology chart also takes place during this step. Thus, the entire Forensic Chronology chart is reformulated into a completed spreadsheet that is chronologically ordered and color coded, and in which the medical chart data is interdigitated with the PDMP data. So, the QME now has the ability to see in the resultant Forensic Chronology chart what drugs were prescribed at what times relative to what the patient was going through in real life. To exemplify, two actual, completed, “real-life” Forensic Chronology charts, which have been redacted to protect the privacy of the persons involved, are attached herewith as Addendum 1 and Addendum 2. The entries at the bottom right side of the Forensic Chronology chart (see FIG. 1 and Addendum A) represent the PDMP “buckets”, e.g., how many pills were prescribed, for how many days, what was the Morphine Equivalent Dose (MEQ), what was the date the prescription was written, were there any refills, and who was the prescribing doctor. The entries at the bottom left side of the Forensic Chronology chart represent the medical record data. The color codes are chosen arbitrarily. For the purpose of the invention herein, the color codes were: pink or tangerine is for hospital or emergency room visits; yellow is for imaging; blue is for the specific target doctor, Dr. T, and also for prescriptions written by Dr. T; pink is for a doctor other than the target doctor (e.g., a specialist, or a doctor the patient was seeing prior to coming to the target doctor), including prescriptions written by other than the target doctor; green is for drug screen including UDT; and white is for the nursing notes. The resultant completed Forensic Chronology document that is thus obtained becomes the source document for the subsequent steps of the method herein. Each patient's Forensic Chronology document provides the chronology of events as they unfolded timewise, on the basis of which the target doctor (Dr. T) can reasonably be held as the doctor responsible for making decisions based on how things had been progressing with the patient. This step also constitutes the beginning of the evaluation process of the target doctor (Dr. T), as the QME would now start being able to notice from patients' Forensic Chronology charts any possible aberrations, patterns, or events that are “outside the usual course of medical practice”. For example, the color-coded data is a graphic way to see whether the patient is “doctor-shopping”; if the patient is getting narcotics right in the middle of care being provided by the target doctor (Dr. T), and the target doctor has the ability to pull that patient's PDMP data, which means that the target doctor knows that the patient is getting narcotics elsewhere, yet the target doctor goes ahead and prescribes narcotics to the patient anyway, then it can be safely concluded that the target doctor is prescribing narcotics “outside the usual course of medical practice”. For another example, a patient's data shows that the patient was positive for oxycodone that they were being prescribed, but the data was negative for benzodiazepine that they were also being prescribed, and was also negative for any oxycodone metabolites; what that usually means is that the patient has likely scraped off a tiny bit of the oxycodone while they were in the bathroom and added it to the urine sample, thus the sample showed positive for oxycodone but not for the metabolites, i.e., the patient was not taking the oxycodone, and the target doctor knew that there was a “medication inconsistency”, or should have paid attention to that, but did not, because the data was in the chart! Yet, the next day the target doctor went ahead and issued another prescription for oxycodone for the patient anyway! Thus, when the QME starts seeing inconsistencies and patterns of aberrancy, that is when the review of observations starts being documented and summarized into the “Review Observations” space of the Forensic Chronology chart. (For examples, see the “Review Observations” space in Addendum 1 and Addendum 2). And all the risk factors start being added by the QME into the Forensic Chronology chart:

Illegal drug history, mental health co-morbidities, physical health co-morbidities, alcohol history, smoking history; pain diagnosis in the patient's own words, actual diagnosis that the target doctor added, the Bates number, and the current medications that are being prescribed by the target doctor. Thus, it is upon the completed Forensic Chronology chart that the whole foundation of the QME's opinion rests. Once the completed Forensic Chronology chart is in hand, the QME now has three things that are brought into play as the foundation for what follows. (1) The first is the chronology, i.e., knowing the series of events as they unfolded over time, according to which the target doctor (Dr. T) can now reasonably be held responsible for making decisions based on how things were progressing with the patient. (2) The second is that the completed Forensic Chronology Chart constitutes the first iteration of summarizing the data upon which a well-founded opinion can be based, as the “Review Observations” and the “Conclusions” fields of the chart (see Addenda 1 and 2) have been filled out by the QME based on the events that the QME has gleaned from the Forensic Chronology chart. (3) The third is that the Forensic Chronology chart constitutes the source document based upon which the next levels of summarization will be built.

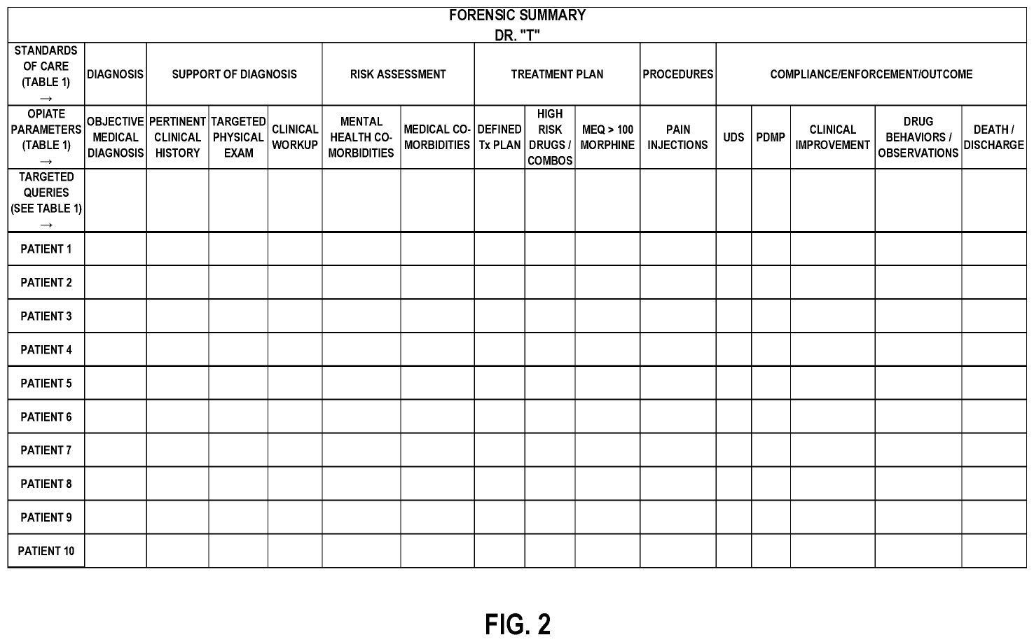

Once the completed Forensic Chronology chart is in hand, it is then used by the QME as the source document to generate the second in the series of three sequential work products, the Forensic Summary chart (or spreadsheet). The Forensic Summary summarizes the Forensic Chronology. Thus, the QME creates a “skeleton” Forensic Summary chart of the invention based on the Standards of Care criteria (see FIG. 2 and Addendum B). In the top row of the Forensic Summary chart are listed the six Standards of Care, in the second row are listed the fifteen Opiate Parameters, and in the third row are listed the Targeted Medical Chart Queries (see Table 1 above). In the leftmost column of the Forensic Summary chart are listed, one per row, all the patients that are part of the investigation of the target doctor (Dr. T) (i.e., all the patients for whom complete Forensic Chronology charts have been generated in the previous step, one per each) (for example, see Addendum 3). (It is understood that other alternative arrangements and layouts of the Forensic Summary chart are possible that would lead the QME to the same observations and conclusions). Thus, the QME begins translating the findings in the patients' Forensic Chronology charts into the patients' respective rows in the Forensic Summary chart. This is done by determining whether standards of care in the chart headings have been fulfilled or violated, and the conclusions are input manually for each of the patients into the appropriate cells in the Forensic Summary chart. The general standards of care as defined by the practice of medicine are: making a diagnosis, support of the diagnosis based on the exam and history, doing a risk assessment, formulating a treatment plan, performing (sometimes) pain procedures, and evaluating the patient outcome (compliance, enforcement of aberrant behavior, overall clinical outcome). So, the QME reviewing the patients' Forensic Chronology charts now fills out the Forensic Summary chart based on the fifteen Opiate Parameters (i.e., Pain Management Parameters), using the Targeted Queries (which are the questions that the QME asks under each of the 15 Opiate Parameters). In other words, the data in each patient's Forensic Chronology chart is evaluated by the QME based on the general standards of care, the pain management concepts, and the specific questions related to those pain management questions. Color coding is used in this chart as well, as a visual aid, at the same time that the QME is inputting their determinations. Once the color-coded Forensic Summary chart is completed, it represents the next level of summarizations and conclusions of the QME. The completed Forensic Summary chart shows the specific patient findings relative to the 15 standards of care/opiate parameters. The completed color-coded Forensic Summary chart becomes the “go-to” document that the QME uses to write their Executive Summary. Thus, in filling out the Forensic Summary, the QME is essentially taking raw data in the Forensic Chronology charts and expressing this data in accord with Standards of Care criteria, phrased in a very succinct manner. As an example, a completed “real-life” Forensic Summary chart, which has been redacted to protect the privacy of the persons involved, is attached herewith as Addendum 3.

Next, the QME uses the completed color-coded Forensic Summary chart in drafting their Executive Summary relative to the target doctor (Dr. T). The QME is thus able in the Executive Summary, which is a narrative summary, to formulate an opinion that carries with it a very high degree of certainty. The Executive Summary reflects the major events that are documented in the Forensic Summary that find their roots in the exhaustive evaluation of the medical charts and the PDMP data.

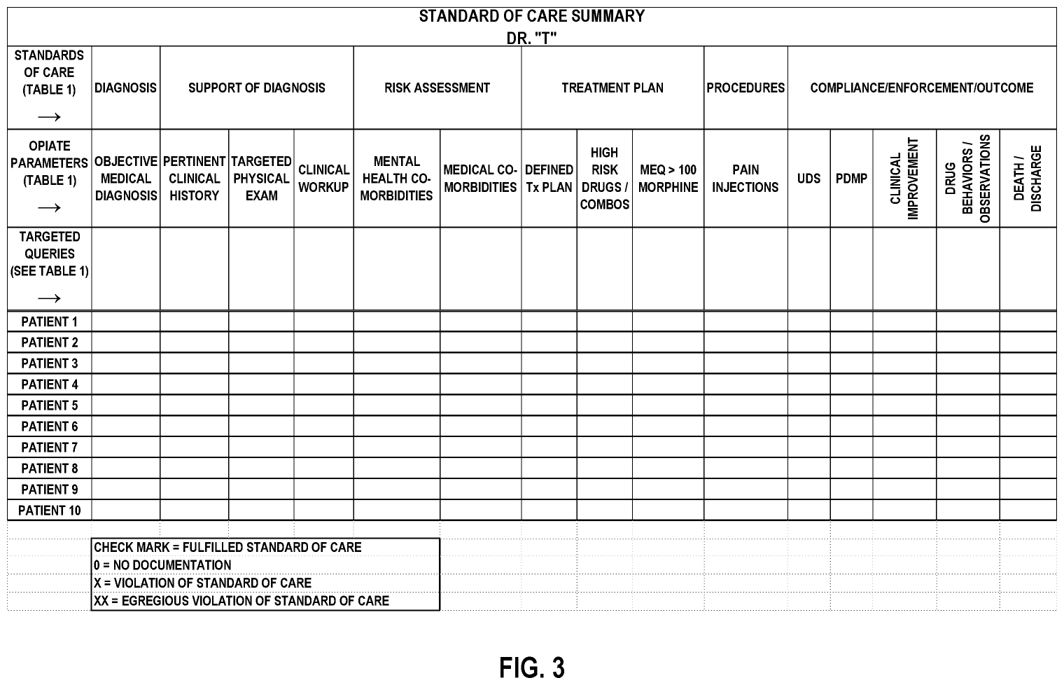

Next, or concurrent with the development of the Executive Summary, the QME converts the information found in the completed Forensic Summary chart into the third in the series of three sequential work products, the Standard of Care Summary chart. The Standard of Care Summary chart summarizes the Forensic Summary chart further, typically into a single page document. Thus, the QME creates a “skeleton” Standard of Care Summary chart based on the Standards of Care criteria (see FIG. 3 and Addendum C). Then, in the “skeleton” Standard of Care Summary chart, the QME expresses each of the observations in the Forensic Summary via a notation: A check mark if the Standard of Care is fulfilled, a zero if there is no data, an X if there is a violation of the Standard of Care, and a XX if there is an egregious violation of the Standard of Care such that the ultimate outcome could have been avoided had that Standard of Care been observed. Alternatively, instead of starting out with a “skeleton” Standard of Care Summary chart, the Standard of Care Summary chart may be prepared by making a copy of the completed Forensic Summary chart, then replacing the observations in the Forensic Summary chart copy with the foregoing notations. A completed “real-life” Standard of Care Summary chart, which has been redacted to protect the privacy of the persons involved, is attached herewith as Addendum 4. This particular real-life example in Addendum 4 indicates, without reasonable doubt, that the target doctor (Dr. T) has consistently not prescribed controlled substances in a legitimate medical manner that meets standards of care, i.e., has been consistently issuing controlled substance prescriptions “outside the usual course” and/or “for other than legitimate medical use”.

Thus, at the conclusion of the steps of the method disclosed herein, the QME would have in hand the completed three work products, Forensic Chronology, Forensic Summary, and Standard of Care Summary, which are ready to be used, e.g., in prosecution of suspected prescribers of controlled substances as irrefutable evidence that said prescribers of controlled substances have operated “outside the usual course” and/or “for other than legitimate medical use”. For example, these documents (objective methodology) may be used by the QME (or the prosecutor), when called on to testify in court, as irrefutable, objective evidence, beyond reasonable doubt, of criminal activity by the prescribers of controlled substances, and may be used to pursue criminal prosecution.

In another embodiment, a method is disclosed herein for developing the series of three work products, Forensic Chronology, Forensic Summary, and Standard of Care Summary, described in the foregoing. Illustratively, said method comprises the following steps:
(a) create a skeletal Forensic Chronology spreadsheet chart for each patient from a set of patients of a particular targeted medical provider, wherein the fields in said skeletal chart are reflective of the medical data found in the patient's medical chart and the patient's Prescription Drug Monitoring Program (PDMP);
(b) obtain the medical chart for each of the patients in the set of patients in step (a), wherein each patient's medical chart includes all available medical data for said patient;
(c) obtain the PDMP data for each of the patients in step (a);
(d) manually transfer the data for each patient from the patient's medical chart and PDMP into the skeletal Forensic Chronology spreadsheet chart created in step (a) for that patient, resulting in a Forensic Chronology spreadsheet populated with the medical events for that patient;
(e) sort the Forensic Chronology spreadsheet for each patient by date, to obtain a Forensic Chronology spreadsheet in which the medical events are organized in chronological order;
(f) color code the data in each patient's chronologically ordered Forensic Chronology spreadsheet obtained in step (e) according to a predetermined color pattern, to obtain a color-coded Forensic Chronology spreadsheet for that patient;
(g) create a single skeletal Forensic Summary spreadsheet chart organized according to the predetermined criteria of 6 Standards of Care, 15 Opiate Parameters, and Targeted Medical Chart Queries, described in the foregoing and in Table 1 above, and that includes a row for each patient from said set of patients;
(h) manually summarize the data in the color-coded, chronologically organized Forensic Chronology spreadsheets for all of the patients into the single Forensic Summary spreadsheet, and color-code the data;
(i) create a skeletal Standard of Care Summary spreadsheet chart organized according to the predetermined criteria of 6 Standards of Care, 15 Opiate Parameters, and Targeted Medical Chart Queries, described in the foregoing and in Table 1 above;
(j) translate the data in the color-coded Forensic Summary spreadsheet in step (i) by inputting the data into the Standard of Care Summary spreadsheet chart using the notations “check mark” for compliance, “0” for no data, “X” for violation of Standard of Care, and “XX” for egregious violation of Standard of Care;
(k) obtain the completed Standard of Care Summary spreadsheet chart;
wherein the predetermined color pattern in step (f) is according to the following: blue for prescribed medications and office visits by the particular medical provider, pink for other-provider consultations and prescriptions, green for UDS, purple for imaging, white for nurses' notes, and orange for hospital and ER visits.

In another embodiment of the invention, disclosed herein is a training method for physicians, medical providers (and the like), regarding the standards of medical care in general, and particularly concerning how to consistently prescribe controlled substances in a legitimate manner that meets generally accepted medical standards of care, i.e., in a manner that is not “outside the usual course” and/or “for other than legitimate medical use”. Said training method includes the step of teaching said physicians and medical providers the 6 Standards of Care, 15 Opiate Parameters, and Targeted Medical Chart Queries described in the foregoing and in Table 1 above, and the manner by which to consistently prescribe controlled substances in a legitimate manner that meets said Standards of Care.

### Additional Discussion

The Applicant himself is a highly qualified medical expert witness, being a medical doctor with several decades of medical practice, including in the fields of anesthesiology, pain management, and addiction. He has a lengthy record of working with the Drug Enforcement Administration (DEA), Federal Bureau of Investigation (FBI), Food and Drug Administration (FDA), Department of Justice, municipal/state courts, and state medical boards. He aids in the criminal prosecution of pill mills and over-prescribing practitioners suspected of issuing controlled substance prescriptions “outside the usual course”, and “for other than legitimate medical purposes”.

In the following, a discussion of the evolution of the method of the invention is presented. Up to this time, there have been no objective published criteria that the Applicant is aware of to determine without reasonable doubt whether a prescriber of controlled substances is prescribing for legitimate medical use or not. There are some generally understood concepts of medicine. However, if someone were to ask an expert in this field whether a medical chart represents a legitimate use of medicine, the typical response would be subjective and opinion-based, rather than objective and data-based. According to the Applicant's experience in the field, based on what they have seen and read and the many other testimonies they have attended and learned from, the response to the foregoing question would be along the lines of, for example, “well, she was selling her medication, and she was picked up by the police, and she's on a really high dose, and she's got it in combination with some of these other drugs that are fairly common in terms of what we see for street use and euphoria; no, I don't think it's legitimate medical use.” Thus, the response seems to be possibly biased or “shot-gunned”.

The invention herein evolved slowly during Applicant's involvement as an expert witness in the context of criminal litigation, fraud investigation, pill mill analysis, insurance review, and clinical practice, pertaining to prescriptions of controlled substances. The invention has been field tested for efficacy and legal foundation, and improved incrementally. It has been recently highly modified to meet the requirements of legal prosecution, court acceptability, and medical accuracy, and has evolved into its current format.

The Applicant started out many years ago, hired by the Indiana Attorney General's office, at a time when no existing objective methodology was available to clearly identify criminal activity of rogue and/or careless prescribers of controlled substances. The Applicant would be charged with determining whether certain prescribers of opiates were issuing prescriptions for legitimate medical purposes in the usual course of medical practice; and the Applicant was expected to review medical charts and ultimately objectively answer that question. At the initial stages, a superficial methodology was followed by the Applicant, obtaining and examining the prescription data of patients and looking at the medications that were being prescribed and the effects that they were having on the patients, the risk-benefit ratio of the medications and the patient's clinical response, and observing data that shows that the patient's condition is not improving, but rather that the patient's problems were getting worse. Over time, the Applicant began to glean criteria and forming them into more objective measurements, forming “buckets' that were then used to go through the patients' medical charts, and to present these various buckets to make the legal arguments. At the early stages of development of the invention, the Applicant would look at the medications being prescribed and the dose of the medications, and convert them to morphine equivalents (e.g., see: https://en.wikipedia.org/wiki/Equianalgesic), knowing that there was a linear relationship between morphine equivalents and overdose. Accordingly, morphine equivalents became one objective criterion in the fledgling method. Another objective criterion that was introduced later was to look for polypharmacy, pertaining to combinations of drugs that are extremely euphoric, addictive, and/or have unique adverse effects and profiles. Thus, it was determined that polypharmacy combinations and their risk factors can be used as another criterion.

At a later point, the CDC and various other governmental agencies and pain management peer organizations, as well as the published literature, began putting out clinical observations indicating which patients were more likely to get addicted than not, and it became apparent that mental health was a major issue in controlled substance abuse; and the Applicant started specifically looking at which mental health illnesses were associated with addiction overdose, and considering that mental health was a risk factor that a prescribing doctor should know about, from the patient's medical chart, and should take into account. It became clear that the mental health diagnoses are important to be looked at. Also, the general health of the patient, even though it seemed to be self-evident, was not being taken into consideration, and was not being put into an objective set of criteria. For instance, if one examined the death certificates on addicted people, one would see that there was a commonality, cardiopulmonary failure (i.e., the addicted person has stopped breathing, and their heart stops), which is the endpoint for overdose and addiction. So, it stood to reason that if a patient comes in to see the doctor and has COPD, and is a smoker, and has respiratory depression, then it is more likely that this patient will have fewer reserves, and thus is more liable to an overdose. So, that becomes a risk factor that one would expect the doctor foundationally to define before they decide to prescribe controlled substances, such as opiates. Accordingly, a risk factor that is termed herein as “physical co-morbidities” was included; it would include any respiratory problems, cardiac problems, kidney (renal) problems, and liver (hepatic) problems, figuring that any deficiencies in any of those areas is going to cause a problem with “physiologic reserves” and to how a patient might respond to further respiratory depression, how the patient might cope with further cardiopulmonary depression, and how the patient was going to metabolize and clear the drug. For instance, in the case of liver function, it is known that the liver metabolizes 95% of drugs, so any liver dysfunction should be taken into consideration by the prescribing physician. Thus, the developing method herein evolved by addition of a review of the end effects to the patient as a risk factor: what does the controlled substance do to the heart in terms of depression, what does it do to the respiratory system in terms of depression, how is the drug metabolized and cleared via the liver and the kidney? Those became co-morbid risk factors that needed to be considered. Those became some of the physiologic and medical aspects or factors that the prescribing doctor should be expected to consider and should be held accountable for. The goal was to recognize the concept that pain management is what is called herein a “bio-psychosocial issue”, which means that addiction and aberrant behaviors are end effects of a combination of biological issues, psychological issues, and social issues. So, if one were to go back and examine what the profiles are of the people who are likely to get addicted, biologically one would come to understand that some people get addicted because they are given too much narcotic. For instance, it might be somebody who goes in for hernia repair and they are given too much Percocet, and thus they are iatrogenically started on the road to addiction just because they were given too much medication; that is the biological component. The psychological component would have to do with a patient who has an underlying psychiatric or mental health issue, depression, anxiety, schizophrenia, PTSD, bipolar (or other anxiety disorders). Those are the types of patients that would generally be associated with the psychological part of the biopsychosocial spectrum; those are the individuals who try to cope with life and are prone to addiction because of mental illness comorbidities. Prior addiction would be another psychological issue (and not necessarily addiction to drugs, but it can be addiction to nicotine, alcohol, or any sort of an addictive diagnosis that is accepted by the medical community and coded in the Diagnostic and Statistical Manual of Mental Disorders (DSM-5.). So, the prescribing physician would be expected to look at past history of all addictive issues of a patient as part of the psychological evaluation. The social component would be recognition of the fact that among addicts there is a disproportionate number who are economically deprived, who are very poor, do not have jobs, on the lower end of the social ladder, and thus are more likely to develop an addiction of any sort, particularly to drugs. Also, it is known that preadolescent individuals who have undergone any kind of abuse (sexual, emotional, or physical abuse) are statistically prone to develop addiction to multiple substances (drugs, alcohol, nicotine). Likewise, recognizing that divorce, family dysfunctional interactions, “adult abuse” (either on the male or female side), are social factors that would contribute to the proclivity for addiction as a way to cope. Accordingly, the biopsychosocial model was integrated by the Applicant into the method, and became a part of the risk factors. The prescribing doctor should be expected to consider the biopsychosocial issues of a patient as part of their foundational workup. If it is determined that the prescribing doctor has ignored or did not consider the biopsychosocial issues of a patient, yet went ahead and prescribed the controlled substance, or if they have prescribed the controlled substance despite knowing that biopsychosocial issues are present, then this prescribing doctor should be scored as one who did not prescribe the medications for a legitimate medical purpose.

In addition to the risk factors in terms of co-morbidity, mental health, and the biopsychosocial factors, according to which controlled substance prescribing physicians are scored in the method herein, another major area, definition of the practice of medicine, contributed to the development of the method herein. The practice of medicine is 3-fold: Examine a patient; formulate a diagnosis; and institute a treatment plan. A licensed physician in the United States, in the course of carrying out their duties, is expected to follow this foundational 3-fold approach when examining a patient. After the physician sees the patient for the first time, subsequently it becomes an iterative process. The physician sees the patient again after a period of time (say, a few weeks), re-examines the patient, re-assesses their diagnosis, and assesses the treatment plan to see if the treatment plan is working. As part of the method herein, a review is made as to whether the patient did get examined by the physician. If so, one looks at the physical exam and history (past medical history, work history, social history), and at the documentation of the examination. Then one looks to see if the physician has made a proper diagnosis. Following is an example: A patient has come in to the doctor complaining of a low back pain (note: 80% of complaints in pain medicine have to do with low back pain). The physician needs to examine and translate the complaint into an objective medically founded diagnosis. So, the physician examines the patient, checks the patient's reflexes, does a couple provocative maneuvers, orders an MM, and checks the past medical history to see what other doctors may have done. The physician is expected, in making the diagnosis, to take into consideration all of these factors. For instance, the physician may diagnose and determine that the low back pain is caused by a herniated disk or sciatic nerve root compression, or some other deformity. Or is the diagnosis simply a subjective repetition of the patient's chief complaint, lacking any objectivity or support of examination? Then the question becomes, did the physician formulate an objective, legitimate and acceptable diagnosis for the use of chronic opiates, supported by examination and testing, and accepted by the general medical community as one that is reasonably treated with long-term controlled substances? Are there other factors that the physician has considered? Is the back pain caused by musculoskeletal factors, such as, for example, the patient being overweight, a smoker, aerobically deconditioned, presence of social stresses in the home or workplace, or marital stressors. The physician would be expected to look at these factors and determine that the patient's problem may not require the use of chronic narcotics. If the physician, being one who is suspected of prescribing narcotics for monetary purposes, sees the patient for a short visit (for example, 5 minutes), fails to perform an adequate examination, but prescribes narcotics, then the methodology herein would flag the situation and conclude that opiates are not being used for a legitimate medical purpose. This example illustrates how the method herein would need to consider the diagnosis and determine whether the narcotic prescription was for a legitimate medical purpose. The next thing as part of the method herein would be to review the treatment plan instituted by the doctor. The treatment plan should not be de facto treatment with opiates or other narcotics; based on past medical history and current diagnosis, the treatment plan should not be repetitive and should be the most conservative/least harmful option. Alternative conservative treatment options might include weight loss, cessation of smoking, improvement of eating habits, aerobic exercise, physical therapy, acupuncture. Appropriate treatment options should be considered before starting chronic opiate therapy. According to the method herein, one looks to see if the treatment plan has been looked at from a more conservative stance to begin with, and then expanded as necessary depending upon how the patient responded and cooperated? Then, this becomes an iterative process. If the patient, for instance, has not been cooperating with the treatment plan, e.g., has not lost weight, continues to eat unhealthily, and nothing has changed in their habitus, etc., then it is not appropriate for the doctor to prescribe narcotics. Very often a medical chart may show that the physician apparently does not care; the patient demands a narcotic, and they get it, which is totally inappropriate. So, according to the method herein, one considers the patient's cooperation, the patient's outcome, any aberrant behaviors on the part of the patient; because besides how the patient may or may not be responding to whatever the treatment plan is that the physician offered, the question also has to be asked: what is the patient doing outside the medical clinic and outside work and outside home? Are they selling their drugs? Is there evidence that the police picked the patient up for dealing? Is there evidence that their spouse is also going to the physician and getting the same narcotics for a similar diagnosis of low back pain? etc.? Also, if one reviews the prescription summary obtained from the PDMP and finds out that the patient is obtaining the same drug from another physician, that is a clear indication that the patient is doctor shopping. So, the behaviors that the patient demonstrates become important, and become specific criteria that must be looked at and be placed in yet another “bucket” to see if one can ultimately define whether the physician is doing their job. Since the patient's chart has presumably been put together by the physician, it is reasonable to assume that everything that is in the chart is what the physician looked at foundationally to make their decision whether to prescribe narcotics. By definition, a medical chart record is the compilation of all the materials, observations, examinations, and tests that went into formulating a diagnosis and a treatment plan. If an outside observer, e.g., a medical expert witness, looks at the same medical chart documentation the physician looked at and provided as foundational for their care of the patient, and the outside observer comes up with a different conclusion with regard to the legitimacy of the use of narcotics, then it is fair to hold the physician accountable for things expected of them that they did not do that would be normal standard of care, whether it is omission as well as commission.

As can be easily understood from the foregoing, the basic concepts of the present invention may be embodied in a variety of ways. The invention involves numerous and varied embodiments described herein of analyzing the medical chart, providing the series of three sequential work products, leading to the objective conclusion whether controlled substances are being prescribed “outside the usual course” and/or “for other than legitimate medical use”. As such, the particular embodiments or elements of the invention disclosed by the description or shown in the figures accompanying this application are intended to be exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It is to be understood that, as used herein, the grammatical conjunction “and/or” refers throughout to either or both of the stated possibilities.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Moreover, for the purposes of the present invention, the term “a” or “an” entity refers to one or more of that entity unless otherwise limited. As such, the terms “a” or “an”, “one or more” and “at least one” can be used interchangeably herein.

The terms “chart” and “spreadsheet” are used interchangeably herein, and refer to a worksheet made up of rows and columns that help sort and arrange data, as is traditionally and commonly understood.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed, or deemed to be admitted as prior art with respect to the invention.

The claims set forth in this specification are hereby incorporated by reference as part of this description of the invention, and the applicants expressly reserve the right to use all of or a portion of such incorporated content of such claims as additional description to support any of or all of the claims or any element or component thereof, and the applicants further expressly reserve the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicants do not waive any right to develop further claims based upon the description set forth above as a part of any continuation, division, or continuation-in-part, or similar application.

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following claims are desired to be protected. It will be apparent to one of ordinary skill in the art that various changes and modifications can be made to the claimed invention without departing from the spirit and scope thereof. In addition, all publications cited herein, if any, are indicative of the level of skill in the art and are hereby incorporated by reference in their entirety as if each had been individually incorporated by reference and fully set forth.

What is claimed is:

1. A method useful for objectively determining the medical legitimacy of controlled substance prescriptions issued by a medical provider who is a prescriber of controlled substances to a group of patients, wherein the method is based on the data in the medical charts and in the Prescription Drug Monitoring Program (PDMP) for each patient in the group of patients, and wherein the method comprises preparation of a coded chronologically organized Forensic Chronology Excel spreadsheet for each patient in the group of patients, said method comprising the following steps:
(a) create a skeletal Forensic Chronology Excel spreadsheet for each of the patients in said group of patients, wherein the row labels and column headings in said skeletal Forensic Chronology Excel spreadsheet are reflective of the dates and corresponding medical data and clinical events found in the patient's medical chart and the patient's PDMP;
(b) obtain the medical chart for each of the patients in said group of patients, wherein each patient's medical chart includes all available medical data for said patient;
(c) obtain the PDMP data for each of the patients in said group of patients, wherein each patient's PDMD data includes all available prescription drug data for said patient including controlled substance prescribing;
(d) carry out manual summarization of the data for each patient from the patient's medical chart and PDMP, and manually transfer the summarized data into that patient's skeletal Forensic Chronology Excel spreadsheet created in step (a), thus resulting in a populated Forensic Chronology Excel spreadsheet for each patient;
(e) sort by date the populated Forensic Chronology Excel spreadsheet from step (d) for each patient, to obtain a chronologically organized Forensic Chronology Excel spreadsheet for each patient, demonstrating an integrated timeline of medical care and controlled substance prescribing;
(f) code the data in each patient's chronologically organized Forensic Chronology Excel spreadsheet obtained in step (e) according to a predetermined coding system, to obtain a coded chronologically organized Forensic Chronology Excel spreadsheet for each patient;
(g) obtain and study copies of the regulations that describe the generally accepted standards of care and pain management concepts of the practice of medicine in the particular state or jurisdiction of said medical provider;
(h) review the medical data and drug data in said coded chronologically organized Forensic Chronology Excel spreadsheet from step (f) for each patient in view of the generally accepted standards of care and pain management concepts of the practice of medicine in the particular state or jurisdiction of said medical provider, and note any possible aberrations, patterns, or events that are outside the usual course of medical practice in said particular state or jurisdiction.

2. The method of claim 1, wherein the manual transfer of the data in step (d) is done via transcription and/or cutting and pasting.

3. The method of claim 1, wherein the coding system of the data in step (f) comprises the use of color codes.

4. The method of claim 3, wherein the use of color codes is according to a predetermined color pattern that demonstrates the type of clinical event involved.

5. The method of claim 4, wherein the predetermined color pattern that demonstrates the type of clinical event involved is the following: prescribed medications and office visits with the target medical provider are colored blue; other-provider consultations and prescriptions are colored pink; UDS is colored green; imaging is colored purple; nursing notes are colored white; and hospital and emergency room visits are colored orange.

6. The method of claim 1, wherein the coding in step (f) is carried out before carrying out the sorting by date in step (e).

7. The method of claim 1, wherein said method further includes preparing a Forensic Summary Excel spreadsheet regarding said medical provider who is prescribing controlled substances, comprising the following steps:
(a) create a skeletal Forensic Summary Excel spreadsheet that includes column headings representing generally accepted medical standards of care and pain management;
(b) immediately under said column headings in (a) insert another row of subheadings that subdivide further the generally accepted medical standards of care and pain management according to applicable care standards and parameters for the use of opiates;
(c) label the rows below the heading and subheading rows in (a) and (b) by listing all the patients in said group of patients, one per row;
(d) further review the medical data and drug data in the coded chronologically organized Forensic Chronology Excel spreadsheet for each patient, and determine whether the generally accepted medical standards of care and pain management and the parameters for use of opiates have been fulfilled or violated;
(e) manually input concise entries describing all the determinations from step (d) into the appropriate skeletal Forensic Summary Excel spreadsheet cells for each patient, to obtain a filled-out Forensic Summary Excel spreadsheet.

8. The method of claim 7, wherein said method further includes inserting immediately below the row of subheadings in step (b) an additional row of subheadings defining targeted queries pertaining to controlled substance standards, wherein said targeted queries assist in the determination of whether the generally accepted medical standards of care and pain management and the parameters for use of opiates have been fulfilled or violated.

9. The method of claim 7, wherein said method further includes the step of coding of the Forensic Summary Excel spreadsheet cells according to a predetermined coding system.

10. The method of claim 9, wherein the coding system includes the use of color codes as a visual aid.

11. The method of claim 7, wherein the generally accepted medical standards of care and pain management in the column headings include the following: Diagnosis, Support of Diagnosis, Risk Assessment, Treatment Plan, Procedures, and Compliance/Enforcement/Outcome.

12. The method of claim 7, wherein the generally accepted medical standards of care and pain management are those specific to the particular state or jurisdiction of said medical provider.

13. The method of claim 7, wherein said method further includes preparing a Standard of Care Summary Excel spreadsheet that is a concise and objective summary of the medical legitimacy of controlled substance prescriptions issued by said medical provider, said method comprising the following steps:
(a) create a copy of said filled-out Forensic Summary Excel spreadsheet;
(b) further review the entries in the filled-out Forensic Summary Excel spreadsheet and/or the Forensic Chronology Excel spreadsheet of each patient and determine the degree and/or severity of any violations of the standards of care and the parameters for use of opiates;
(c) replace the cell entries of the copy of the filled-out Forensic Summary Excel spreadsheet by expressing them with notations that reflect the degree and/or severity of violations of the standards of care and the parameters for use of opiates;
and, thus, obtain said Standard of Care Summary Excel spreadsheet.

14. The method of claim 13, wherein said notations include the following: check mark=standard of care is fulfilled; 0=documentation is lacking; X=violation of the standard of care; and, XX=egregious violation of the standard of care.

15. A Forensic Chronology spreadsheet that is obtained according to the method of claim 1.

16. A Forensic Summary spreadsheet that is obtained according to the method of claim 7.

17. A Standard of Care Summary spreadsheet that is obtained according to the method of claim 13.
